(19) United States
(12) Patent Application Publication
(10) Pub. No.: US 2021/0236840 A1
(43) Pub. Date: Aug. 5, 2021

(54) COMBINED SONODYNAMIC AND PHOTODYNAMIC THERAPY TO TREAT DEEP SEATED CANCER AND OTHER HYPERPOLIFERATIVE DISEASE AND ULTRASOUND AND LIGHT TRIGGERED RELEASE OF NEAR INFRARED PHOTOSENSITIZERS AND CHEMOTHERAPY AGENTS FROM POLYACRLAMIDE BASED NANOPARTICLES

(71) Applicants: Photolitec, LLC, East Amherst, NY (US); Health Research, Inc, Buffalo, NY (US)

(72) Inventors: Ballav Borah, North Tonawanda, NY (US); Ravindra Pandey, Williamsville, NY (US); Joseph Cacaccio, Buffalo, NY (US); Farukh Durrani, Buffalo, NY (US); Adam Sumlin, Getzville, NY (US); Eric Kauffman, Buffalo, NY (US)

(21) Appl. No.: 16/777,765

(22) Filed: Jan. 30, 2020

(51) Int. Cl. A61N 5/06; A61N 7/00; A61K 41/00

(52) U.S. Cl. CPC A61N 5/062; A61N 7/00; B82Y 5/00; A61N 2005/0659; A61K 41/0076

(57) ABSTRACT

New advantageous compositions and methods for treatment of malignancies such as basel cell carcinoma and other skin cancers, brain tumors, head and neck cancer, kidney cancer, prostate cancer and other cancer types. The new compositions and methods may also be used for treatment of non-cancer problems such as dermatological diseases, sexual diseases, anti-fungal and antibacterial and wound healing. A new photodynamic therapy/sonodynamic therapy (PDT/SDT) combination approach using near infrared (NIR) tetrapyrrolic photosensitizers (PSs) with long wavelength absorption, in the range of 750-800 nm, that provides deeper tissue penetration. Such combinations may be postloaded onto PAA nanoparticles (NPs) for better delivery to a tumor site. A preferred sonodynamic compound is fullerene 60 but other sonodynamic compounds may be employed. The unique NP formulations of the invention can contain chemotherapeutic agents releasable by ultrasound in cancer or other hyperproliferative tissue. NP formulations may also contain tumor targeting moieties.

### BACKGROUND OF THE INVENTION

Photodynamic therapy (PDT) is widely used for the treatment of a variety of cancers. It requires a tumor-avid photosensitizer (PS), oxygen and light of appropriate wavelength. For a typical PDT treatment, PS is injected to patients intravenously and after a certain time (depending from PS to PS), the PS shows significantly higher accumulation in tumors than the surrounding cells. The tumor is then exposed to light at the optimal tumor-uptake, which excites the PS and releases the energy before coming to ground state. The energy reacts with the oxygen present in tumors and converts it into singlet oxygen (¹O₂), and/or other reactive oxygen species (ROS), which destroys the tumors.

PDT works well for several indications, but it suffers from certain limitations: (a) Depth of light penetration limits the depth of activation. (b) Sufficient light needs to reach the tumor in order to activate PS and the formation of ROS responsible for the tumor kill. Therefore, efforts have been underway in order to develop PS with near-infrared (NIR) absorption in the range of 750-800 nm that has a deeper penetration than those currently in use.

It has recently been shown that limited depth of penetration might be resolved by using ultrasound (US) waves to activate the drug at deeper depths.

Sonodynamic therapy (SDT) is an emerging approach that involves a low-intensity ultrasound and specialized chemical agents known as sonosensitizers. Unlike photodynamic therapy (PDT), which has poor light tissue penetration, ultrasound can penetrate deeply into tissues to mediate the cytotoxicity of sonosensitizers. It can be focused into a small region of a tumor to activate the tumor-avid agent(s) and offers the possibility of non-invasively eradicating a variety of tumors including glioblastoma and kidney cancer, where PDT shows limitations.

Similar to photodynamic therapy (PDT), the generation of reactive oxygen species (ROS) has been proposed as the main biological occurrence that leads to the cytotoxic effects, which is achieved via the synergistic action of two components, i.e. the energy absorbing sonosensitizers and ultrasound (US), which are otherwise harmless if used alone. Despite some promising results, a lack of investigation, in designing effective sensitizers, mechanisms behind US sonosensitizer-mediated ROS generation (using ultrasound instead of light), and efficient delivery of the agent at the tumor-site, has prevented SDT from reaching its full potential.

Current understanding of the mechanism of sonodynamic therapy (SDT) suggests that sonosensitization is due to the chemical activation of sonosensitizers inside or in the close vicinity of hot collapsing cavitation bubbles, to form sensitizer-derived free radicals either by direct pyrolysis or due to reactions with —H and —OH radicals, formed by pyrolysis of water. These free radicals (mostly carbon-centered) react with oxygen to form peroxyl and alkoxyl radicals. Unlike —OH and —H, which are also formed by pyrolysis inside cavitation bubbles, the reactivity of alkoxyl and peroxyl radicals with organic components dissolved in biological media is lower and hence, have a higher probability of reaching critical cellular sites. Sonodynamic therapy appears to be a promising modality for cancer treatment since ultrasound can penetrate deep within the tissue, and can be focused in a small region of tumor to chemically activate relatively non-toxic molecules (e.g. some porphyrins). SDT could thus have advantages in treating certain tumor-types.

It is known that some of the tumor-avid porphyrin-based compounds used for PDT can also be used for SDT, and a combination approach has been limited to treating localized cancer due to limited depth of penetration of the photosensitizer (PS). Further, in such systems, Reactive oxygen species (ROS) do not yield as much singlet oxygen as desired.

Initial study suggests that most of the NIR PSs (with singlet oxygen yield in the range of 30-50%, e.g. HPPH, for the treatment of head & neck cancer, esophagus, lung cancers and photobac with absorption and emission wavelength>790 nm for the treatment of glioblastoma) can also be used for the treatment of cancer by SD. However, in contrast to PDT, SD does not require NIR PS. The major requirements for effective sonosensitizers are (a) high singlet oxygen or other reactive oxygen producing ability, (b) tumor retention ability, and (c) tumor-specificity.

A further problem with cancer treatment is that when chemotherapy is used, the active compound must usually be systematically injected thus causing significant damage to otherwise healthy tissue. Attempts have been made to cause the chemotherapeutic agent to be selectively absorbed by the tumor but even then, some of the active agent can also be absorbed by healthy tissue. Certain PDT agents (especially porphyrin related compounds) are selectively absorbed and until activated do not cause cellular damage. Most other effective chemotherapeutic agents, however, are immediately active upon injection.

### BRIEF SUMMARY OF THE INVENTION

The invention includes new and advantageous compositions and methods for a broad range of malignancies such as basel cell carcinoma and other skin cancers, brain tumors, head and neck cancer, kidney cancer, prostate cancer and other cancer types. The new compositions and methods may also be used for treatment of non-cancer problems such as dermatological diseases, sexual diseases, anti-fungal and antibacterial and wound healing.

The invention includes a new photodynamic therapy/sonodynamic therapy (PDT/SDT) combination approach using sonosensitizers and near infrared (NIR) photosensitizers (PSs) with long wavelength absorption, in the range of 750-800 nm, that provides deeper tissue penetration. Such sonosensitizers may be postloaded onto polyacrylamide (PAA) nanoparticles (NPs).

Further an approach of the invention is to use NIR PS with higher reactive oxygen species (ROS) producing ability, which is effective for both types of treatment, e.g. bacteriopurpurinimide (photobac) conjugated with zinc or palladium.

The invention further includes a combination of a tetrapyrrolic photosensitizer with a sonosensitizer. A preferred sonosensitizer is fullerene C60.

In accordance with the invention, it has further been unexpectedly discovered that polyacrylamide type nanoparticles can carry tetrapyrollic photosensitizers across the blood brain barrier. This has been demonstrated by optical Imaging and Fluorescence imaging of the tetrapyrollic HPPH photosensitizer (PS) in PAA nanoparticles (NPs) at a dose 0.47-0.75 mmol/kg in Normal Balb/c mice showing PS uptake in Brain.

In addition, the unique NP formulations of the invention can contain chemotherapeutic agents until they reach the cancer or other hyperproliferative tissue where they can be released by ultrasound.

In accordance with the present invention, it has now been discovered that there is an improved result when ultrasound treatment is fractionated, i.e. is provided in relatively close spaced time intervals until the dose limit has been reached rather than continuously. Fractionated SDT in combination with new near infrared (NIR)-PDT (near 800 nm) and fullerenes (C60 moieties) with and without nanoparticle constructs for treating cancers, e.g. glioblastoma and kidney cancer is novel.

The invention includes:

A composition including a photosensitizer and a sonosensitizer where the photosensitizer preferably includes a near infrared tetrapyrrolic photosensitizer and the sonosensitizer preferably includes a fullerene. The fullerene is most preferably fullerene C60 and the photosensitizer most preferably is a tetrapyrrolic metal conjugate and the metal is desirably Zn++. The tetrapyrrolic photosensitizer preferably includes photobac but may be another tetrapyrrolic compound such as a chlorin, bacteriochlorin, phthalocyanine, pheothiazine, pyropheophorbide, or other purpurinimide and their chelates and peripheral substituents.

In accordance with the invention, the photosensitizer and sonosensitizer are postloaded onto a PAA nanoparticle.

The invention also includes a method for treatment of deep seated malignancy including the steps of introducing a near infrared photosensitizer and a sonosensitizer into the malignancy and exposing the malignancy to light at a wave length that activates the photosensitizer and to ultrasound at a frequency that activates the sonosensitizer.

The photosensitizer and sonosensitizer used in the method may be the same compound and may be a metal conjugated photobac.

The invention further includes the method where a chemotherapeutic agent is also postloaded onto the nanoparticle and where the chemotherapeutic agent may be releasable into the malignancy by ultrasound. The nanoparticle may also contain a cancer targeting moiety.

### BRIEF DESCRIPTION OF THE SEVERAL VIEWS OF THE DRAWINGS

FIG. 1 shows a treatment flow chart for personalized treatment using ultra-sound and/or light triggered release of near-infrared (NIR) photosensitizers and/or chemotherapy agents from polyacrylamide-based nanoparticles, use of these compounds in cancer imaging and therapy with and without nanoparticles. Showing treatment schemes utilizing treatment methods to obtain complete remission. PDT=photodynamic Therapy; PET-ONCO=Positron emission tomography Commonly using 18F-fluoro-2-deoxyglucose (FDG) resulting in excellent resolution of tumors of even small size. SDT=sonodynamic therapy. Methods may be sequential or simultaneous.

FIG. 2 shows a setup for determining ultra-sound (US) exposure and US-triggered release.

FIG. 3 shows Scheme 2, for post-loading of PSs/chemo agents in amine functionalized polyacrylamide (AF-PAA) nanoparticles.

FIG. 4 shows US-triggered Release of (a) HPPH (PS), (b) HPPH-Zn (PS), (c) Photobac (PS), (d) Photobac-Zn (PS), (e) Doxorubicin (Chemo), (f) Curcumin (Chemo) and (g) Photobac-Zn & Fullerene C60 (PS) Post-loaded Functionalized Nanoparticles. US-triggered Releases were determined in 1% Has-PBS. Dispersions were made to a final HPPH (ε: 47,500 Lmol⁻¹cm⁻¹ at 661 nm), HPPH-Zn concentration (c: 40,873 Lmol⁻¹cm⁻¹ at 650 nm), Photobac (c: 41,032 Lmol⁻¹cm⁻¹ at 785 nm), Photobac-Zn (c: 55,190 Lmol⁻¹cm⁻¹ at 790 nm), Doxobubicin (c: 10,550 Lmol⁻¹cm⁻¹ at 495 nm) and Curcumin (c: 55,000 Lmol⁻¹ cm⁻¹ at 426 nm) of 20 μM and US irradiated using a 2.0 w/cm², 1 MHz. Samples were irradiated for 5, 10 and 20 mins, filtered through a 100 KDa centrifuge filter, and filtrate absorbance was read on an optical density spectrophotometer.

FIG. 5 shows UV-Vis spectra of (i) original, (ii) retentate, (iii) filtrate of HPPH (a-c) and Doxorubicin (d-f) post-loaded Cationic PAA NPs after 5, 10 and 20 mins of US irradiation respectively at 2.0 W/cm², 1 MHz.

FIG. 6A shows In vivo PDT response of HPPH alone.

FIG. 6B shows PDT-SDT combination response of HPPH in cationic NPs @ 0.47 umol/kg in SCID mice bearing U87 Glioma xenografts. The combination PDT-SDT unexpectedly shows a 60% complete response compared to a 37% complete response for PDT alone.

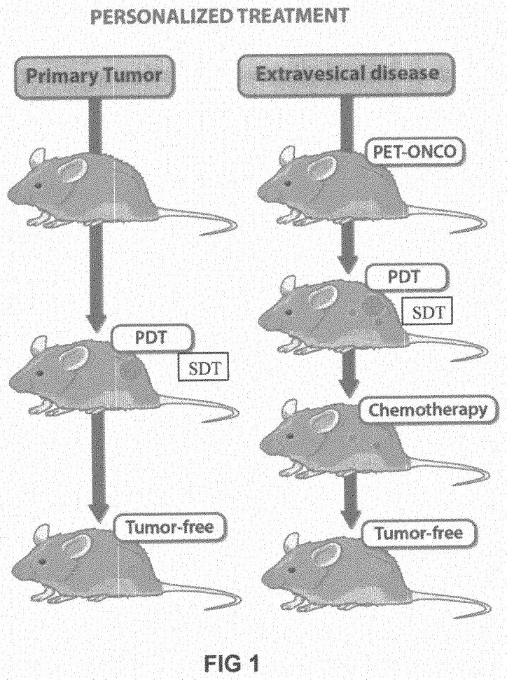

### DETAILED DESCRIPTION OF THE INVENTION

Preferred photosensitizers are tetrapyrrolic photosensitizers such as pyropheophorbides, purpurinimides, chlorins, bacteriochlorins, phthalocyanines, pheothiazines, HPPH (2-(1-Hexyloxyethyl)-2-devinyl pyropheophorbide-a) and Photobac (bacteriopurpurinimide).

Conjugation of certain metals (e.g., Zn or Pd) to NIR PS (e.g. Photobac) further enhances its singlet oxygen producing ability. “Conjugated” or “conjugate” as used herein means associated by a covalent bond or by a weak bond such as a hydrogen bond or chelate.

Interestingly, certain fullerenes (C60 molecules), non-porphyrin-based compounds, not suitable for treating deeply seated tumors with absorption near 400 nm, with limited depth of penetration by light, but due to its >90% singlet oxygen producing ability, could be an excellent candidate under optimized SDT treatment parameters. In an initial attempt to develop a dual treatment (SDT+PDT) modality agent, C60 fullerene was conjugated to a NIR PDT agent to obtain that result. In accordance with the invention, such conjugates have been found to have excellent sonodynamic properties.

While C60 fullerene is a preferred sonosensitizer, other fullerenes could be employed. Fullerenes in general are allotropes of carbon whose molecule consists of carbon atoms connected by single and double bonds so as to form a closed or partially closed mesh, with fused rings of five to seven atoms.

Additionally or optionally, other sonosensitizers could be used in conjunction with the photosensitizer, e.g. xanthene based compounds, certain non-steroidal anti-inflammatory compounds, and certain dye structures such indocyanine green and acridine orange. Specific sonodynamic compounds that may be used are erytho sin B (EB), rose Bengal (RS), sparfloxacin (SPFX), levofloracin (LVFX), lomefloxacin (LFLX), curcumin, indocyanine green, (ICG), acridine orange (AO), hypocrelian B, and 5-aminolevulinic acid (5-ALA).

In addition, the invention includes unique polyacrylamide (PAA) nanoparticle (NP) formulations where the photodynamic and sonodynamic compound(s) of the invention are postloaded onto the nanoparticle, i.e. after the nanoparticle is initially formed. “PAA” as used herein is intended to include modified polyacrylamide where the amide group is functionalized and/or acrylamide is copolymerized with another monomer. A compound having a functionalized amide group may be obtained by direct functionalization or by any other synthetic route. The nanoparticles can contain chemotherapeutic agents until they reach the cancer or other hyperproliferative tissue where they can be released by ultrasound.

The nanoparticles can also contain targeting agents. Examples of such targeting agents include peptides, antibodies, carbohydrates and small molecules known for their targeting to a variety of cancer types.

Most of the sensitizers developed for the use in SDT and PDT are hydrophobic in nature with limited water solubility. On the basis of past experience, we extrapolate that these compounds will be formulated either in (a) 0.5% Tween80/D5W, (b) 2% Pluronic-127 and (c) polyacrylamide (PAA) NPs.

The use of ultrasound and light triggered multifunctional nanoparticles designed for cancer-imaging and combination therapy (SDT/PDT) in addition to chemotherapy helps to treat the localized cancer and metastases.

These NP formulations for combination therapy are advantageous because (i) their hydrophilicity and charge can be altered, (ii) they possess enormous surface area that can be modified with functional groups possessing a diverse array of chemical and biochemical properties, including tumor selective ligands, and (iii) due to their subcellular and sub-micron size, they can penetrate deep into tissues and are generally taken up efficiently by cells.

### Design of Ultra-Sound (US) Exposure Apparatus:

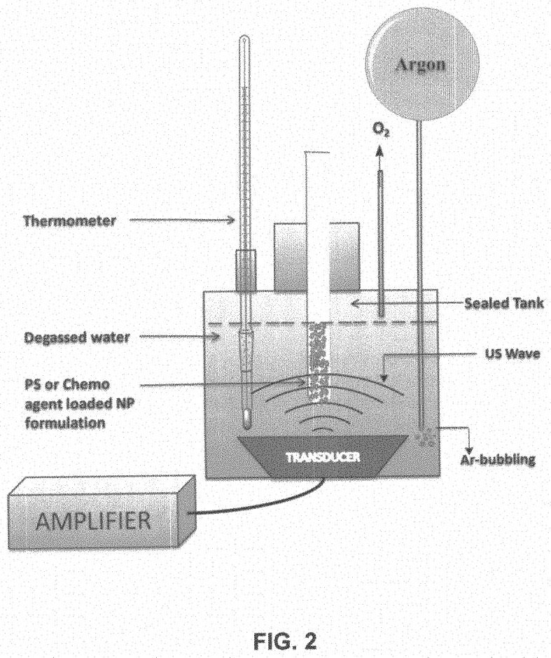

An Ultrasound design used in accordance with examples of the invention is shown in FIG. 2. The 1.00 MHz continuous focused ultra-sound (US) transducer is a circular single disk with diameter of 42.5 mm and a point target focus (PTF) of 76 mm. The transducer is submerged in distilled degas sed water at the bottom of a sealed glass setup with upward standing wave. The design is a novel closed/sealed setup with continuous argon (Ar) gas bubbling to degas oxygen throughout the experiment, unlike current in-vitro setups available. This set up allows for the US wave to spread uniformly and with minimal scattering and energy loss. A thermometer is also incorporated for monitoring the temperature to increase consistency. Two small ports for Ar-gas bubbling and to remove dissolved oxygen are also employed. The system is preferably a closed system. A second apparatus may be used to hold standard culture plates to expand the type of in vitro experiments under the same or similar parameters.

### Post-Loading of Photosensitizers and Chemotherapeutic Agents in Amine Functionalized PAA Nanoparticles:

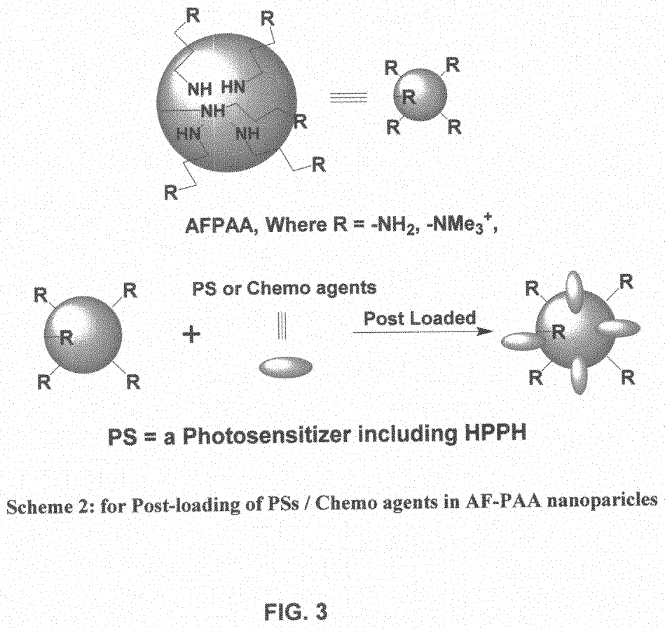

FIG. 3 shows a scheme for Post Loading of PSs/chemotherapeutic agents in amine functionalized, e.g. polyacrylamide, nanoparticles (AF-PAA). PAA as used herein is intended to include functionalized polyacrylamide. Various amine functionalized nanoparticles and their preparation are discussed in U.S. Patent Publication 2005/0196343 published Sep. 8, 2005 for U.S. Patent Application 60/548,105 filed Feb. 28, 2005 to Reddy et al., incorporated herein by reference.

Lyophilized AF-PAA nanoparticles were dispersed in 1% Tween-80 PBS (pH 7.4, 10 mM) to a final concentration of 10 mg/mL. PSs or chemo agents were dissolved in DMSO to a final concentration of 20 mM, 20 μL of this mixture were added to 2 mL of nanoparticle solution and was stirred for 2 hours. The nanoparticle dispersion were transferred to Amicon Ultra-4 100 kDa centrifuge filter and centrifuged at 4,000 rpm for 80 minutes to remove excess DMSO, Tween-80, and PSs/Chemo agents that did not post-load. The absorbance of the filtrate was measured, and if signal for the photosensitizer was detected, the retentate was reconstituted to the original volume with fresh PBS and re-centrifuged.

This was continued until no signal was detectable in the filtrate, spectrophotometrically. The nanoparticle solutions were syringe-filtered through a 0.2μ filter and stored at 4° C. Concentration could not be measured with nanoparticles in ethanol suspension, as the scatter skewed the absorbance measurement. The concentration was measured by mixing an aliquot of suspension in ethanol, and centrifugation in a benchtop centrifuge at 14,000 RPM for 1 minute to pellet and remove emptied nanoparticles.

### Ultra-Sound (US)-Triggered Release

The cationic PAA NPs post-loaded with photosensitizer (HPPH or other photosensitizers) and chemotherapeutic agents (Doxorubicin, Curcumin etc) were mixed with 1% (w/v) Human Serum Albumin in phosphate buffer solution (PBS). The absorbance spectrum of the solution was measured spectrophotometrically and was marked as the original absorbance. The US-triggered release was measured at 2.0 W/cm² at 1 MHz irradiation for 5 mins, 10 mins and 20 mins using a SDT apparatus shown in FIG. 2. The temperature during the entire duration of irradiation was monitored by Omega Microprocessor Thermometer (Model HH23, Type J-K-T Thermocouple) and was found to be constant. The solutions after the ultrasound (US) irradiation were then centrifuged in an Amicon Ultracel-4, 100 kDa centrifuge filter at 4,000 RPM for 15 minutes. The filtrate was marked as filtrate #1 and absorbance was measured spectrophotometrically. The retentate was reconstituted to the original volume with 1% HSA PBS, thoroughly mixed with a pipette, and was re-centrifuge filtered. The second filtrate was marked filtrate #2 and absorbance was measured. To measure what was retained by the nanoparticles after the two wash steps, the nanoparticles were reconstituted with 1% HSA in PBS, thoroughly mixed with a pipette and absorbance was measured. The two wash steps were combined together as the total filtrate. This signified the total released, and the absorbance measurement of the retentate indicated the percentage of the post-load that was retained at various time intervals. The results (Table 1-5 min.) show that US-triggered release can be achieved by irradiating for as low as 5 mins at dose rate of 2.0 W/cm²

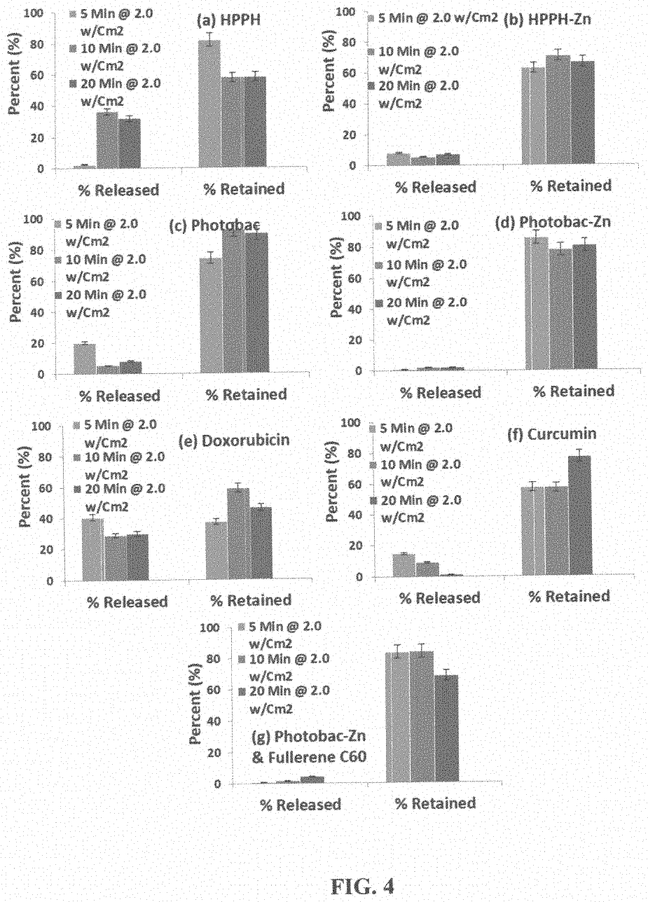

FIG. 4 shows US-triggered Release of (a) HPPH (PS), (b) HPPH-Zn (PS), (c) Photobac (PS), (d) Photobac-Zn (PS), (e) Doxorubicin (Chemo), (f) Curcumin (Chemo) and (g) Photobac-Zn & Fullerene C60 (PS) Post-loaded Functionalized Nanoparticles. US-triggered Releases were determined in 1% Has-PBS. Dispersions were made to a final HPPH (ε: 47,500 Lmol⁻¹cm⁻¹ at 661 nm), HPPH-Zn concentration (c: 40,873 Lmol⁻¹cm⁻¹ at 650 nm), Photobac (c: 41,032 Lmol⁻¹cm⁻¹ at 785 nm), Photobac-Zn (c: 55,190 Lmol⁻¹cm⁻¹ at 790 nm), Doxobubicin (c: 10,550 Lmol⁻¹cm⁻¹ at 495 nm) and Curcumin (c: 55,000 Lmol⁻¹ cm⁻¹ at 426 nm) of 20 μM and US irradiated using a 2.0 w/cm², 1 MHz. Samples were irradiated for 5, 10 and 20 mins, filtered through a 100 KDa centrifuge filter, and filtrate absorbance was read on an optical density spectrophotometer.

TABLE 1
US irradiation of PS (HPPH) post-loaded cationic PAA-NMe₃⁺ NPs.
% Normalized HPPH (ε: 47,500 Lmol⁻¹cm⁻¹ at 661 nm)

| | | Standard | | Standard |
|---|---|---|---|---|
| | % | Error | % | Error |
| | Released | (Filtrate) | Retained | (Retentate) |
| 5 | 2.38 | 0.06 | 82.31 | 0.26 |
| Min | | | | |
| 10 | 36.34 | 0.38 | 58.17 | 2.97 |
| Min | | | | |
| 20 | 32.05 | 0.21 | 58.42 | 0.38 |
| Min | | | | |

*2.0 W/cm2 power, 1.0 MHz

TABLE 2
US irradiation of PS (HPPH-Zn) post-loaded cationic PAA-NMe₃⁺ NPs.
% Normalized HPPH-Zn (ε: 40,873 Lmol⁻¹cm⁻¹ at 650 nm)

| | | Standard | | Standard |
|---|---|---|---|---|
| | % | Error | % | Error |
| | Released | (Filtrate) | Retained | (Retentate) |
| 5 | 8.24 | 0.12 | 63.26 | 0.15 |
| Min | | | | |
| 10 | 5.56 | 0.10 | 71.15 | 0.29 |
| Min | | | | |
| 20 | 7.16 | 0.10 | 67.06 | 2.29 |
| Min | | | | |

*2.0 W/cm2 power, 1.0 MHz

TABLE 3
US irradiation of PS (Photobac) post-
loaded cationic PAA-NMe₃⁺ NPs.

| | | Standard | | Standard |
|---|---|---|---|---|
| | % | Error | % | Error |
| | Released | (Filtrate) | Retained | (Retentate) |
| 5 | 19.74 | 0.23 | 74.50 | 0.25 |
| Min | | | | |
| 10 | 4.98 | 0.67 | 92.30 | 0.23 |
| Min | | | | |
| 20 | 7.90 | 0.06 | 90.10 | 0.66 |
| Min | | | | |

*2.0 W/cm2 power, 1.0 MHz

TABLE 4
US irradiation of PS (Photobac-Zn) post-loaded cationic
PAA-NMe₃⁺ NPs.
% Normalized Photobac-Zn (ε: 55,190 Lmol⁻¹cm⁻¹ at 790 nm)

| | | Standard | | Standard |
|---|---|---|---|---|
| | % | Error | % | Error |
| | Released | (Filtrate) | Retained | (Retentate) |
| 5 | 0.59 | 0.00 | 85.71 | 0.21 |
| Min | | | | |
| 10 | 2.08 | 0.15 | 78.13 | 0.62 |
| Min | | | | |
| 20 | 1.95 | 0.10 | 80.83 | 0.38 |
| Min | | | | |

*2.0 W/cm2 power, 1.0 MHz

TABLE 5
US irradiation of Chemo agent (Doxorubicin) post-loaded cationic
PAA-NMe₃⁺ NPs.
% Normalized Doxorubicin (ε: 10,550 Lmol⁻¹cm⁻¹ at 495 nm)

| | | Standard | | Standard |
|---|---|---|---|---|
| | % | Error | % | Error |
| | Released | (Filtrate) | Retained | (Retentate) |
| 5 | 40.59 | 0.10 | 37.24 | 0.10 |
| Min | | | | |
| 10 | 28.73 | 0.06 | 59.14 | 0.06 |
| Min | | | | |
| 20 | 29.71 | 0.00 | 46.16 | 0.06 |
| Min | | | | |

/cm2 power, 1.0 MHz*2.0

TABLE 6
US irradiation of Chemo agent (Curcumin) post-loaded cationic
PAA-Me₃⁺ NPs.
% Normalized Curcumin (ε: 55,000 Lmol⁻¹cm⁻¹ at 426 nm)

| | | Standard | | Standard |
|---|---|---|---|---|
| | % | Error | % | Error |
| | Released | (Filtrate) | Retained | (Retentate) |
| 5 | 14.61 | 0.23 | 57.73 | 1.04 |
| Min | | | | |
| 10 | 8.70 | 0.12 | 57.36 | 0.25 |
| Min | | | | |
| 20 | 1.08 | 0.01 | 77.53 | 1.95 |
| Min | | | | |

*2.0 W/cm2 power, 1.0 MHz

TABLE 7
US irradiation of PS (Photobac-Zn & Fullerene C60) post-loaded cationic
PAA-NMe₃⁺ NPs.
% Normalized Photobac-Zn (ε: 55,190 Lmol⁻¹cm⁻¹ at 790 nm)

| | | Standard | | Standard |
|---|---|---|---|---|
| | % | Error | % | Error |
| | Released | (Filtrate) | Retained | (Retentate) |
| 5 | 0.80 | 0.32 | 83.05 | 0.20 |
| Min | | | | |
| 10 | 1.35 | 1.62 | 83.49 | 0.31 |
| Min | | | | |
| 20 | 4.18 | 0.00 | 67.94 | 0.12 |
| Min | | | | |

*2.0 W/cm2 power, 1.0 MHz

TABLE 8
DLS (Volume-Wt) in 1% Tween PBS
Cationic PAA-NMe₃⁺ NPs

| Diameter (nm) | 11.0 nm | 52.7 nm |
|---|---|---|
| Std. Dev (nm) [%] | 0.7 nm [6.6%] | 3.6 nm [6.9%] |
| Percent | 99.0% | 1.0% |

Doxorubicin post-loaded cationic PAA-NMe₃⁺ NPs

| | | |
|---|---|---|
| Diameter (nm) | 11.0 nm | 66.1 nm |
| Std. Dev (nm) [%] | 0.7 nm [6.6%] | 9.3 nm [14.0%] |
| Percent | 95.4% | 4.6% |

### Photophysical Characterization:

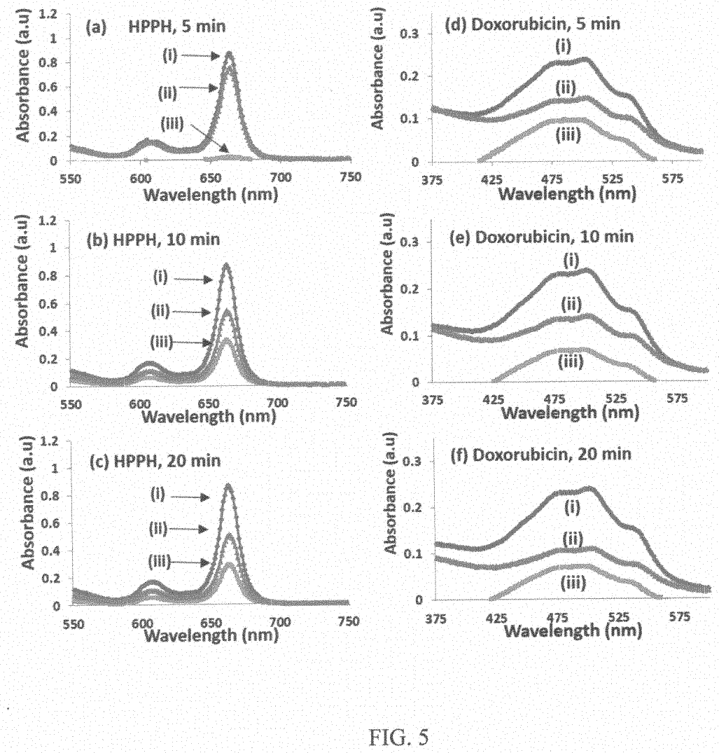

FIG. 5 shows UV-Vis spectra of HPPH (a-c) and Doxorubicin (d-f) post-loaded Cationic PAA NPs after 5, 10 and 20 mins of US irradiation respectively. UV-Vis absorption spectra of the compounds (MeOH solutions) were acquired using a Varian (Cary-50 Bio) spectrophotometer. The extinction coefficients of all compounds were determined by weighing a particular amount of solid and dissolving in a 50 ml volumetric flask using methanol as the solvent. First, the molar concentration (C) of each solution was calculated from its weight and volume. The absorbance (A) obtained from the UV-Vis spectrophotometric measurements was used to determine the extinction coefficient (c) from Beer-Lambert's Law (A=εdC), where d is optical path length (cuvette thickness, d=1 cm).

To calculate the concentration of PS within NPs, the PS post-loaded NP is diluted in ethanol and measured spectrophotometrically using a Varian (Cary-50 Bio) with a molar extinction coefficient (c) of 47,500 L/(mol*cm). To remove the scattering in the absorbance spectra, the NPs were centrifuged filtered with a Microfuge membrane-filter (NANOSEP 100K OMEGA, Pall Corporation) at 5,000 rpm for 10 minutes. The NPs are retained above and the PS loaded within the NP is in the filtrate. The concentration of the PS in filtrate and in NP was measured spectrophotometrically. For measuring the concentrations of Doxorubicin in NPs, the NP solutions were diluted in 200-proof ethanol and measured spectrophotometrically as discussed above using 10,550 L (mol*cm) as the molar extinction coefficient values in methanol.

### Dynamic Light Scattering (DLS)

Table 8 shows DLS of (a) PAA-NMe₃⁺ (cationic) Nanoparticles in 1% Tween-80 PBS 52.7±3.6 nm (Volume-Wt NICOMP distributions) and (b) post loaded Doxorubicin on PAA-NMe₃⁺ (cationic) 66.1±9.3 nm. 10 mg/mL PAA-NMe₃⁺ (cationic) NPs were dispersed through vortexing and sonication in 1% Tween-80 PBS. They were filtered through a 0.2 μm syringe filter and immediately read on a DLS Instrument. The polydispersed samples contained micelles 10-13 nm.

Measurement of hydrodynamic size provides information about the solvent layer that assembles around the nanoparticles; a larger apparent diameter indicates a greater ability to arrange a corona of solvent molecules around the nanoparticle, and such nanoparticles are likely to have a higher Zeta potential and are more likely to remain dispersed over time. The dynamic light scattering measurements were performed on a Nicomp 370 Submicron Particle Sizer (Nicomp, Santa Barbara, Calif.). The NP solution was placed in a borosilicate glass capillary tube, and diluted with water to an intensity reading of 300 kHz. The readings were performed in triplicate with each run set for 5 minutes. DLS was performed on loaded and unloaded nanoparticles dispersed in 1% Tween-80 water to determine their hydrodynamic diameter. NICOMP proprietary software was used to measure the sizes of different populations in the polydispersed samples. Samples were syringe filtered through a 0.2 micrometer filter and immediately analyzed. Two runs of 5 minutes each were collected. The volume-weighted analysis was chosen as the most representative measurement of hydrodynamic size.

### Example 1

It has been reported that in comparison to free-base, the insertion of certain metals (e. g., Zn(II) and PD(II)) in porphyrin system increases their ROS producing ability under ultrasound (US) exposure. However, insertion of metal exhibits blue shift in the electromagnetic spectra thus limiting depth of tissue penetration. Instead of using porphyrins, reduced porphyrin systems (Photobac) in which the pyrrole rings diagonal to each other were reduced with long absorption near 800 nm. Insertion of Zn(II) to Photobac, in accordance with the invention, surprisingly produced red shift instead of blue shift and showed enhanced singlet oxygen producing ability on exposing either with light (photons) or ultrasound (SD).

### Example 2

Use of nanoparticles for efficient delivery of PDT agents: It has been previously shown that PAA (polyacrylamide)-based nanoparticles (NPs) can be used as efficient delivery agents by postloading the desired imaging and therapeutic agents to tumors, with a significant increase in tumor-specificity. The tumor-specificity can further be improved by modifying the peripheral substituents, and/or the incorporation of targeting functionalities at the periphery. Substituted polyacrylamide nanoparticles are intended to be included in the PAA abbreviation. Such “substituted” structures are intended whether or not they result from direct substitution or by some other synthetic route.

Among the SDT agents investigated, fullerenes (C60 compounds) have shown particular interest due to their high singlet oxygen producing ability under SDT treatment conditions. In an attempt to develop dual treatment modality agents, C60 moiety was post-loaded either alone or in combination with Zn(II)-Photobac in PAA NPs, and the stability of the constructs by following the release kinetics approach was determined. Surprisingly, in accordance with the invention, it has now been found that under ultrasound and light treatment parameters, an efficient release of both fullerene and Zn(II) Photobac from the NPs, has now been observed. These results are exciting and provide an enormous potential for the delivery and local release of the desired agents in tumor.

### Example 3

A Zn(II)-bacteriochlorin was conjugated with fullerene and its photophysical properties were determined. Fluorescence maxima was surpriobserved in the long-wavelength region>800 nm of the spectrum (broad). The quantum yields of the generated singlet oxygen were determined to be 35%.

### Example 4

In vitro PDT efficacy (MTT assay): Tumor cells were incubated with the desired sensitizer at variable concentrations (after 4 and 24 h incubation of the sensitizers, the cells were washed and exposed to an appropriate wavelength of light. Finally the treated cells were seeded in 96 well microplates and then incubated at 37° C. for 24 h. Cytotoxicity was determined using MTT assay. Optical density (OD) at 490 nm was then measured using Gen-5 CHS 207 softwere. Killing rate was calculated using the following equation: Killing rate (%): (OD control group-OD treatment group)/OD control group x 100%.

### Example 5

In vitro SDT efficacy (MTT assay): A similar approach as discussed for in vitro-PDT efficacy was used. However instead of light, the cells were exposed with US of various intensities (1-2 W/cm²). U87 (glioma) and RCC7860 (kidney) cell line were treated by light and US, and a significant in vitro cell kill was observed.

### Example 6

Analysis of anticancer efficacy of Photobac was injected in tumor-bearing mice and treated with the laser light at 787 nm. Tumor cure was monitored for 60 days post-laser treatment. At similar treatment parameters, excellent tumor response was observed in female SCID mice and 4/5 mice (CR=80%) and 4/7 male mice (CR=60%) were found to be tumor free up to 60 days As suggested by the United States FDA in a pre-IND meeting, these results were included to the Photobac-PDT-IND submission to FDA. Photobac is being developed for the treatment of glioblastoma by Photolitec, LLC (a spin-off of RPCI) has received an ORPHAN DRUG STATUS from FDA.

### Example 7

Long-term tumor cure by Photobac-PDT on treating nude mice (female) bearing U87 tumors (shoulder) at variable drug doses. The tumors were exposed with light (787 nm, 135 J/cm², 75 mW/cm²) at 24 h post injection. Comparative long-term cure of Photobac-PDT treatment in both and female SCID mice bearing U87 tumors (shoulder) under similar treatment parameters (drug dose: 0.75 μmol/kg; light dose: 135 J/cm², 75 mW/cm².

### Example 8

Photobac-PDT causes vascular shutdown: To investigate the consequences of in vivo PDT treatment, when the tumors were approximately 65 mm³ in volume (BALB/C mice with Colon 26 tumors, Photobac dose: 0.25 μmol/kg and (nude mice/U87, 3 mice/group, Photobac dose: 0.75 μmol/kg), the tumors were treated with a laser light at 787 nm at a dose of 135 J/cm² and 75 mW/cm² at 24 h post-injection of the PS. Light control only group did not receive Photobac, and untreated controls received no treatment. Twenty four hours post laser treatment, mice were euthanized, tumors removed and fixed in zinc. Tissue sections were stained for CD31 (PECAM-1) which is the most widely used endothelial marker for studying angiogenesis/neovascularization. CD31 positive vessels exhibit a brown colored stain as indicated by Histopathology Analysis.

### Example 9

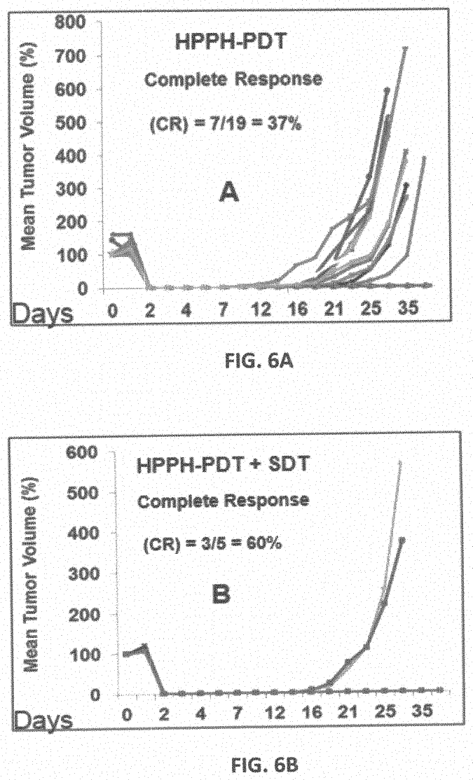

It has been determined that SDT causes tumor necrosis. In PDT, effective agents also show significant tumor necrosis after being exposed to light, and in general, a direct correlation between the degree of necrosis and long-term PDT efficacy has been observed. Activity of HPPH-SDT and Zn(II)HPPH-SDT, in accordance with the present invention, has now been applied to treating breast cancer in SCID mice bearing 4T1 breast xenografts (drug dose: 0.47 μmol/kg, US: fractionated dose, power 1 MHz) From the results summarized in FIGS. 6A and 6B, it is evident that compared to HPPH free-base the corresponding Zn(II) analog, with higher singlet oxygen producing ability shows intense tumor-necrosis. The in vivo studies were performed following the IACUC approved protocol (Pandey: 537M).

What is claimed is:

1. A composition comprising a photosensitizer and a separate sonosensitizer.

2. The composition of claim 1 where the photosensitizer comprises a near infrared tetrapyrrolic photosensitizer and the sonosensitizer comprises a fullerene.

3. The composition of claim 2 where the fullerene is fullerene C60.

4. The composition of claim 2 where the photosensitizer comprises a tetrapyrrolic metal conjugate.

5. The composition of claim 3 where the metal is Zn++.

6. The composition of claim 3 where the tetrapyrrolic metal conjugate and the sonosensitizer are postloaded onto a PAA nanoparticle.

7. The composition of claim 4 where the tetrapyrrolic photosensitizer is photobac.

8. The composition of claim 7 where the photo sensitizer and sonosensitizer are postloaded onto a PAA nanoparticle.

9. A method for treatment of deep seated malignancy comprising introducing a near infrared photosensitizer and a sonosensitizer into the malignancy and exposing the malignancy to light at a wave length that activates the photosensitizer and to ultrasound at a frequency that activates the sonosensitizer.

10. The method of claim 1 where the photosensitizer and sonosensitizer are the same compound.

11. The method of claim 9 where the photosensitizer and sonosensitizer are postloaded onto a PAA nano particle.

12. The method of claim 11 where a chemotherapeutic agent is also postloaded onto the nanoparticle.

13. The method of claim 2 where the photosensitizer and ultrasound sensitizer is a metal conjugated photobac.

14. The method of claim 13 where the photosensitizer and ultrasound sensitizer is a zinc II conjugated photobac further conjugated with a fullerene.

15. The method of claim 12 where the chemotherapeutic agent is released into the malignancy by ultrasound.

16. The method of claim 9 where the photosensitizer is a chlorin, bacteriochlorin, phthalocyanine, pheothiazine, pyropheophorbide, or purpurinimide and their chelates and peripheral substituents.

17. The method of claim 11 where the nanoparticle contains a cancer targeting moiety.

18. The method of claim 9 where fractionated SDT is used in combination with new near infrared (NIR)-PDT (near 800 nm).

19. The method of claim 18 where the sonosensitizer comprises fullerene C60.

20. The method of claim 19 where the photosensitizer and sonosensitizer are postloaded onto a polyacrylamide nanoparticle.
